(19) United States
(12) Patent Application Publication
(10) Pub. No.: US 2015/0099670 A1
(43) Pub. Date: Apr. 9, 2015

(54) Method of preparing post-bisulfite conversion DNA library

(71) Applicants: Weiwei Li, Lindenhurst, NY (US); Jessica Li, Lindenhurst, NY (US)

(72) Inventors: Weiwei Li, Lindenhurst, NY (US); Jessica Li, Lindenhurst, NY (US)

(21) Appl. No.: 13/998,141

(22) Filed: Oct. 7, 2013

(51) Int. Cl. C12Q 1/68

(52) U.S. Cl. CPC C12Q 1/6806
USPC 506/26

(57) ABSTRACT

The invention relates to a method of preparing bisulfite-treated DNA library by ligation of adaptors to DNA after bisulfite conversion. The prepared library is suitable for use in sequencing reactions to analyze genome-wide DNA methylation status.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO A MICROFICHE APPENDIX

Not applicable

### BACKGROUND OF THE INVENTION

1. Field of the Invention

The invention relates to a method of preparing a post-bisulfite conversion DNA library by ligation of adaptors to DNA after bisulfite conversion. The prepared library is suitable for use in sequencing reactions to analyze genome-wide DNA methylation status.

2. Description of the Related Art

DNA methylation occurs by the covalent addition of a methyl group (CH₃) at the 5-carbon of the cytosine ring, resulting in 5-methylcytosine (5-mC). DNA methylation is essential in regulating gene expression in nearly all biological processes including development, growth, and differentiation (Laird P W et al: Annu. Rev. Genet. 30, 1996; Reik W et al: Science, 293, 2001). Alterations in DNA methylation have been demonstrated to cause a change in the gene expression. For example, hypermethylation leads to gene silencing or decreased gene expression while hypomethylation activates the genes or increases gene expression. Aberrant DNA methylation is also associated with pathogenesis of diseases such as cancer, autoimmune disorders, and schizophrenia (Baylin S B et al: Nature Clin Pract Oncol Suppl 1, 2005. Richardson B et al: Clin Immunol, 109, 2003. Grayson D R et al: Proc Natl Acad Sci USA, 102, 2005). Thus gene/region-specific or genome-wide analysis of DNA methylation or 5-methylcytosine (5-mC) could provide valuable information for discovering epigenetic markers used for disease diagnosis, and potential targets used for therapeutics.

Several methods such as whole genome bisulfite sequencing (WGBS) (Cokus SJ et al, Nature, 2008; Lister R et al, Cell, 2008) or reduced representation bisulfite sequencing (RRBS) (Meissmer A et al, Nature, 2008) have been used and are still being used for genome-wide DNA methylation analysis. These methods allow unmethylated cytosine to convert to uracil while 5-mC remains the same with bisulfite treatment so that an epigenetic difference turns to a genetic difference, which can then be detected with sequencing. Bisulfite-sequencing is considered a gold standard assay for gene/region-specific and genome-wide determination of 5-mC because of its single-base resolution and genome-wide coverage. However, current methods are still unsatisfiting in practical use: (1) These methods need quite large amounts of DNA (>5 μg) as input material, which cause difficulty to prepare such amount of DNA from many biological samples such as tumor biopsy, embryonic tissues and circulating DNA; (2) These methods need to first fragment DNA and then ligate adaptors to DNA fragments followed by bisulfite conversion. This procedure causes most of the DNA fragments contained in adaptor-DNA fragment constructs to be broken, and thereby forming mono-tagged templates that will be removed during library enrichment. Thus, incomplete coverage and bias occur when performing whole genome bisulfite sequencing. Recently developed post-bisulfite adaptor tagging (PBAT) (Miura F et al, Nucleic Acids Res, 2012) reduced the amount requirement of input DNA material and avoided the formation of mono-tagged templates. However, PBAT processing is complicated and time consuming. Thus, there is still an ample need for establishing a method that overcomes the weaknesses of above methods.

### BRIEF SUMMARY OF THE INVENTION

The present invention described a method that ligates adaptors to bisulfite treated DNA to generate a post-bisulfite DNA library. Such adaptor ligation of a bisulfite treated DNA sample allows for preparation of a bisulfite DNA template that can then be used for deep sequencing with decreased input DNA amount and higher efficiency. By using the method of the present invention, DNA is fragmented to an appropriate size simultaneously during the bisulfite process in a single stranded DNA form. Such fragmented ssDNA can be converted to double stranded DNA (ssDNA) and then ligated with adaptors after the end polishing, thereby enabling the bisulfite DNA library preparation to be much simpler and quicker than currently used methods.

For preparation of bisulfite DNA template used for deep sequencing, the method of this invention comprises the step of: (1). Isolating DNA from a biological sample; (2). Bisulfite treatment of DNA; (3). Conversion of bisulfite ssDNA into dsDNA. (4). End polishing of dsDNA; (5). Two-end ligation with an adaptor and (6). Amplification after purification of ligated DNA.

Unexpectedly the method presented in this invention significantly overcomes the weaknesses existing in the prior technologies and improves sensitivity and efficiency of the prior DNA ligation-bisulfite conversion methods.

### BRIEF DESCRIPTION OF THE DRAWINGS

FIG. 1, shows a diagram of the process for preparing a bisulfite DNA template used for deep sequencing. The process involves: (1). Isolating DNA from a biological sample; (2). Bisulfite treatment of DNA; (3). Conversion of bisulfite ssDNA into dsDNA. (4). End polishing of dsDNA; (5). Two-end ligation with an adaptor and (6). Amplification after purification of ligated DNA.

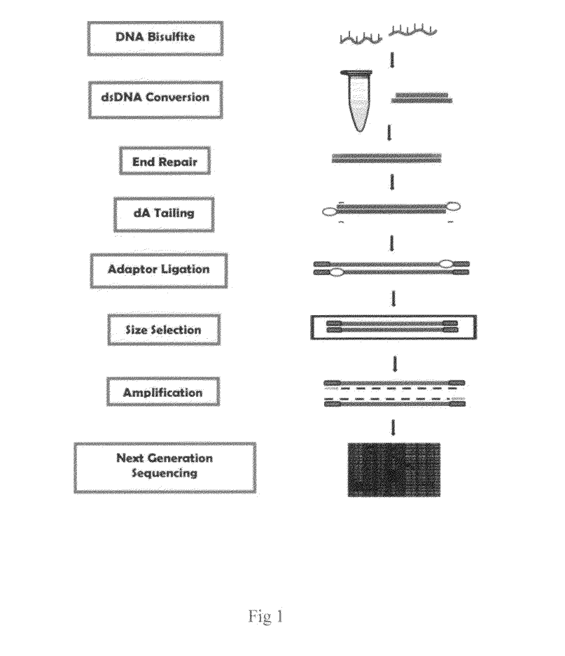

FIG. 2, shows the size distribution of post-bisulfite DNA library fragments by a Agilent bioanalyzer after ligation. FIG. 2A: the size distribution of post-bisulfite (bisulfite-ligation) DNA library; FIG. 2B: the size distribution of ligation-bisulfite DNA library.

FIG. 3, shows qPCR analysis results of the post-bisulfite DNA library and DNA library generated from DNA ligation-bisulfite conversion.

### DETAILED DESCRIPTION OF THE INVENTION

According to the method of this invention, DNA could be isolated by lysis of cells with lysis buffer containing a sodium salt, tris-HC1, EDTA, and detergents such as sodium dodecyl sulphate (SDS). Tissue fragments should be homogenized before lysing. For example, disaggregating of tissue fragments can be performed by stroking 10-50 times, depending on tissue type, with a Dounce homogenizer. DNA can be further purified by mixing with a high concentration of sodium chloride and then adding into a column pre-inserted with a silica gel, a silica membrane, or a silica filter. The DNA that binds to the silica matrix is washed by adding a washing buffer and eluted with TE buffer or water. DNA can also be isolated and purified by using commercially available DNA extraction kits such as QiaAmp blood or tissue kits (Qiagen). The starting materials for DNA extraction can be from various species including, but not limited to, fresh tissues, frozen tissues, formalin fixed and paraffin embedded tissues, body fluids, and cultured cells.

In an embodiment of the invention, the purified DNA sample is treated with bisulfite reagents. These bisulfite reagents may include but not limited to sodium bisulfite, potassium bisulfite, ammonium bisulfite, magnesium bisulfite, sodium metabisulfite, potassium metabisulfite, ammonium metabisulfite and magnesium metabisulfite. Bisulfite salts such as sodium bisulfite or ammonium bisulfite can convert cytosine to uracil and leave the 5-mC the same. Thus after bisulfite treatment 5-mC in the DNA remains the same and unmodified cytosines will be changed to uracil. The bisulfite treatment can be performed by using the methods disclosed in prior art or commercial kits such as the Bisulflash DNA Modification Kit (Epigentek) and Imprint DNA Modification Kit (Sigma). For achieving the optimal bisulfite conversion with desired DNA fragment size for post-bisulfite ligation, the bisulfite reaction should be carried out in an appropriate concentration of bisulfite reagents, appropriate temperature and appropriate reaction time period. A reagent such as potassium chloride that reduces thermophilic DNA degradation could also be used in bisulfite treatment so that the DNA bisulfite process can be much shorter without interrupting a completed conversion of unmethylated cytosine to uracil and without a significant thermodegradation of DNA resulted from depurination.

Once DNA bisulfite conversion is complete, DNA is captured, desulphonated and cleaned. The bisulfite-treated DNA can be captured by a solid matrix selected from silica salt, silica dioxide, silica polymers, magnetic beads, glass fiber, celite diatoms and nitrocellulose in the presence of high concentrations of chaotropic or non-chaotropic salts. The bisulfite-treated DNA is further desulphonated with an alkalized solution, preferably sodium hydroxide at concentrations from 10 mM to 300 mM. The DNA is then eluted and collected into a capped microcentrifuge tube. An elution solution could be DEPC-treated water or TE buffer (10 mM Tris-HCL, pH 8.0, and 1 mM EDTA).

According to the method of this invention, the bisulfite-treated DNA that presents in ssDNA form can be converted into dsDNA through an enzyme-catalyzed DNA strand synthesis in the presence of appropriate primers. The enzymes include but are not limited to DNA-dependent DNA polymerase such as phi29 DNA polymerase, Bst DNA polymerase, exonuclease deficient Klenow DNA polymerase, T4 DNA polymerase, and native and modified T7 DNA polymerase, and RNA-dependent DNA polymerase such as HIV-1 reverse transcriptase, M-MLV reverse transcriptase and AMV reverse transcriptase. The appropriate primers can be selected from various lengths of random primers. These random primers can be from 4 mers to 16 mers, preferably 6-8 mers and most preferably 6 mers (hexamer). In one aspect, the dsDNA is converted from bisulfite-treated DNA using M-MLV reverse transcriptase or AMV reverse transcriptase in the presence of random hexamer. In a further aspect, the dsDNA is converted from bisulfite-treated DNA using exonuclease deficient Klenow DNA polymerase in the presence of random hexamer.

The converted dsDNA from bisulfite DNA fragment samples are repaired by an end repair reaction using methods or kits such as NEB End Repair Kit (New England Biolabs) so that the fragment end is blunt ended. Single nucleotide ‘A’ can then be added to the blunt ended 3′ terminus of each strand of the target fragment duplexes by a reaction with Taq or Klenow exo-polymerase. The fragments are then ligated to adaptors. The adaptors consist of two oligonucleotides and can be partially complementary to be able to form a region of double stranded sequence after annealing. The sequence length of the adaptors can be from 10 nucleotides to 100 nucleotides, preferably from 20 to 60 nucleotides, more preferably from 30-40 nucleotides. The adaptors are ligated to both 5′ and 3′ end of the target oligonucleotide fragments to form adaptor-target constructs. The ligation reaction can be performed by incubating the adaptors and oligonucleotide fragments with ligation enzymes such as T4 DNA ligase. The nucleotide sequence of the adaptors is generally not limited to the invention and may be selected by the user. However the sequences of the individual strands in the non-complimentary region of the adaptors should not exhibit any internal self-complementarity as it could lead to self-annealing or formation of hairpin structures under standard annealing conditions. The adaptor could have a biotin molecule at the 5′ end to enable solid-phase capture of the adaptor-target constructs, for example, onto streptavidin magnetic beads or plates. The adaptor may also include “tag” sequences to mark template molecules derived from a particular source or include non-natural nucleotides.

The ligated samples are purified and size selected to remove unbound adaptor molecules from the adaptor-bisulfite DNA constructs. Any suitable methods can be used to remove excess unbound adaptors. For example, using PCR purification column from Qiagen could help to eliminate unbound adaptors from the samples and running the column-purified samples on 2% certified low range ultra agarose gel can help to select the desired fragment size. The beads-based DNA purification including Agencourt AMPure method is also helpful to remove unbound adaptors. The desired fragment size is from 100-600 bps, preferably 150-400 bps, more preferably 200-300 bps.

Any PCR methods can be used for amplifying the post-bisulfite ligated samples. These methods are known to those of ordinary skill in the art. In general, the PCR reactions can be set up by adding sample, dNTPs, and appropriate polymerase such as Pfu Turbo polymerase, primers and buffer. The PCR is performed for a low number of cycles, for example 15 cycles with a defined temperature and time length for each cycle. The primers (ex: Primer A and Primer B) used for the PCR reaction should be able to anneal to each individual strand of the adaptors ligated at 5′ and 3′end of the adaptor-target-adaptor constructs and is able to be extended in order to generate one complementary to each strand of the adaptor-target-adaptor polynucleotide. Based on the methods of this invention, the unligated DNA fragments will not be amplified even if a few of these fragments are in the samples. Thus only adaptor-bisulfite DNA constructs with intact adaptors on the both ends can be amplified.

Real time PCR can be used for quantifying the post-bisulfite library. The PCR data obtained from the post-bisulfite DNA templates by the methods of this invention can be further analyzed by comparing to the PCR data obtained from a traditional DNA ligation-bisulfite conversion method. Such comparison would further confirm the sensitivity of the method of this invention in 5-mC determination.

The library produced by the PCR amplification can be further purified with various PCR purification methods. These methods including PCR purification kits (Qiagen) are known to those of ordinary skill in the art. The purified library can be further validated by measuring size, concentration and sequence of the library. The size of the library can be determined by running the library on a 2% agarose gel to check whether the size range is as expected. The size of the library can also be obtained by Bioanalyzer analysis. The concentration of the library can be obtained by measuring its absorbance at 260 nm. The representative sequence can be determined by cloning the library into a sequencing vector such as PCR-4Blunt-TOPO (Life Technology) and then sequence individual clones by conventional Sanger sequencing. The library is then used in sequencing to map 5-mC in a genome-wide scale at base-resolution level. The 5-mC and unmethylated cytosines contained in the target portions of the amplified constructs will be read as C and T, respectively during the sequencing. After sequencing, the analysis of 5-mC DNA sequences can be performed by comparing to the known reference genome to map genome-wide 5-mC sites. The 5-mC DNA sequencing data obtained from the prepared DNA templates by the methods of this invention can be further analyzed by comparing to the single base 5-mC DNA sequence data from traditional whole genome bisulfite sequencing in which the DNA library is first constructed followed by bisulfite conversion. Such comparison would further confirm the specificity of the method of this invention in 5-mC determination.

### EXAMPLE 1

The experiment was carried out to generate bisulfite converted DNA. Genomic DNA isolated from human placenta was treated with bisulfite reagents. The bisulfite reaction was set up as follows: DNA sample 10 μl (1 ng -1 μg), 5 M sodium bisulfite solution 120 μl (containing 0.5 M KCl), 6 M NaOH 5 μl. The reaction tubes were placed in a thermal cycler with a heated lid and run with the following program: 4 min at 95° C., 30 min at 65° C., 4 min at 95° C., 30 min at 65° C., 4 min at 95° C., 60 min at 65° C. The sample solution was then mixed with 300 μl of DNA binding buffer and the mixture was added into a spin column, DNA bound on the column was treated with 100 μl of alkali desulphonation solution and then washed with 200 μl of 90% ethanol for twice by centrifugation at 12,000 rpm for 1 min. The DNA was then eluted in 10 μl of Tris buffer. Bisulfite treated DNA was analyzed by real time PCR (42 cycles) with use of modified DNA-specific primers and unmodified DNA-specific primers, which are able to approximately quantify the conversion of cytosine to uracil. The results are shown in Table 1.

TABLE 1
Real-time PCR analysis of bisulfite-treated DNA

| | Cycle Threshold (Ct) | | | | |
|---|---|---|---|---|---|
| Input DNA | 1 | 10 | 100 | 1000 ng | Water |
| β-actin | 34.1 | 31.8 | 27.7 | 23.8 | 42* |
| (modified DNA-specific | | | | | |
| primers) | | | | | |
| GAPDH | 41.2 | 40.5 | 39.5 | 36.9 | 42 |
| (unmodified DNA-specific | | | | | |
| primers) | | | | | |

*no amplification
DNA volume eluted after bisulfite treatment: 20 μμl
DNA volume added in final reaction: 2 μl
Ct^{GAPDH-β-actin} >7 represents 99% of cytosine-thymine conversion

### EXAMPLE 2

The experiment was carried out to generate adaptor-bisulfite DNA constructs.

1. dsDNA conversion: The bisulfite-treated DNA was converted to dsDNA through dsDNA conversion reaction in 0.2 ml PCR tube. The reaction was set up as follows: bisulfite-treated DNA (from 10 ng of input DNA) 10 μl, 5X conversion buffer containing 10 mM dNTP 4 μl, 20 mM random primers 2 μl, Klenow DNA polymerase (5U/ul) 1 μl. Total reaction volume as adjusted to 20 ul by adding an appropriate volume of water. The reaction mix was incubated at 37° C. for 60 mM and was then purified with AMPure XP beads. 36 μl of AMPure XP beads were added into 20 μl of converted DNA solution and incubated for 10 mM. The beads were collected and rinsed by applying the solution to a magnetic field. DNA (>200 bps) was eluted by suspending the beads in Tri-EDTA buffer.

2. End repair: The converted dsDNA were treated with a mixture of enzymes to repair, blunt, and phosphorylate ends. The end-repair reaction was set up as follows: converted DNA fragment 10 μl, 10X end repair buffer (containing dNTP mix 4 mM each) 2 μl, T4 DNA polymerase (3 U/μl ) 1 ul, T4 PNK (10 U/μl) 1 μl. Total reaction volume was adjusted to 20 μl by adding an appropriate volume of water. The reaction mix was incubate at room temperature for 30 min then purified with AMPure XP beads as described in Experiment 1 and eluted in 12 μl DNase/RNase-free water

3. dA tailing: Repaired DNA fragments were incubated with Klenow exo-fragment (3′-5′ exo-) to add a single “A” base to the 3′end. The dA tailing reaction was set up as follows: DNA sample (from previous end repair step) 12 μl, 10X Klenow buffer 2 A dATP (5 mM) 2 μl, Klenow fragment 3′-5′exo- (5 U/μl) 2 μl. Total reaction volume was adjusted to 20 μl by adding an appropriate volume of water. The reaction mix was incubated at 37° C. for 30 min followed by 70° C. for 10 min.

4. Ligation: The dA-tailed DNA fragments were incubated with adaptors to ligate the fragments. Adaptor 1 and adaptor 2 were pre-annealed at a 1:1 ratio. The ligation reaction was set up as follows: DNA sample (from previous dA tailing step) 20 μl, 5X ligation buffer 6 μl, adaptors (at 10:1 molar ratio to DNA sample) 1 μl, T4 DNA ligase (2000 U/μl ) 1 μl. Total reaction volume was adjusted to 30 μl by adding an appropriate volume of water. The reaction mix was incubated at room temperature for 20 min.

5. Size selection: 16 μl of AMPure XP beads were added into 30 μl of DNA-enzyme reaction solution and incubated for 10 mM. The supernatant was collected to a new tube and 7 μl of AMPure XP beads were added into the 46 μl of supernatant and incubated for 10 min. Beads were collected and rinsed by applying the solution to a magnetic field. DNA (>150 bps and <400 bps) was eluted by suspending the beads in Tri-EDTA buffer.

### EXAMPLE 3

The experiment was carried out to specifically amplify and quantify the post-bisulfite DNA library.

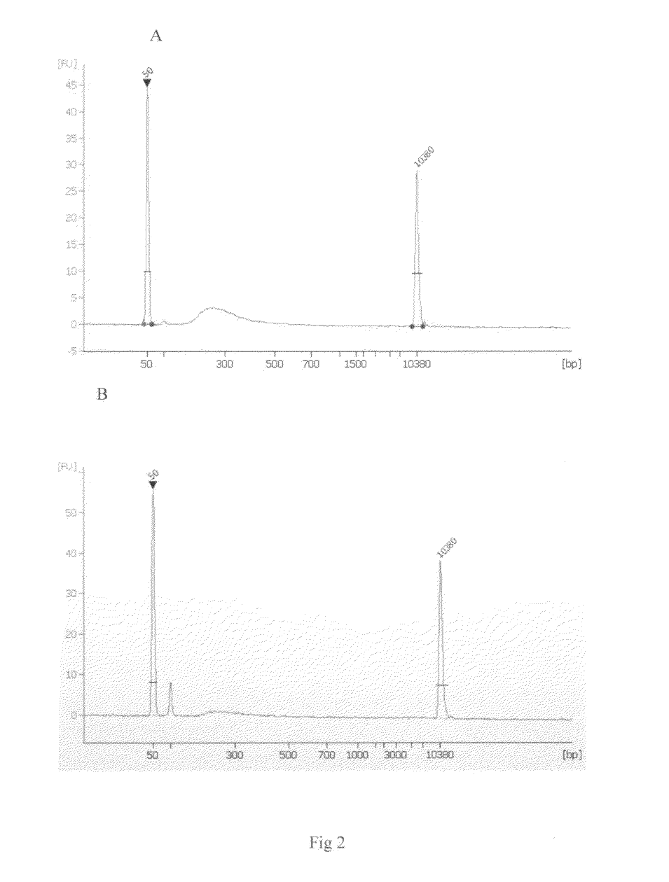

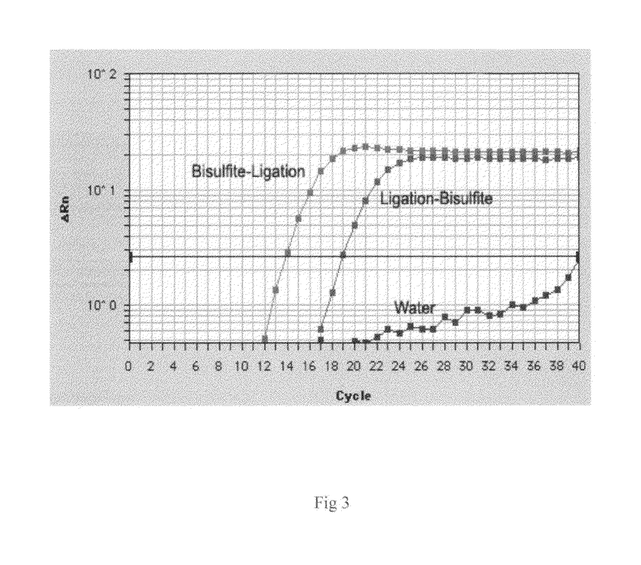

The size-selected DNA sample was amplified by PCR reactions. (a) The PCR reaction was set up as follows: size-selected sample 1 μl, Q5 High-Fidelity DNA Polymerase 1 U, 5X Q5 reaction buffer 4 μl, 10 mM dNTPs 0.4 A forward PCR primer 1 μl, reverse PCR primer 1 μl. Total reaction volume was adjusted to 20 μl by adding an appropriate volume of water. The following PCR protocol was used: 30 sec at 98° C., 18 cycles of 10 sec at 98° C., 15 sec at 55° C., and 20 sec at 72° C. As comparison, the DNA library prepared by ligation of placenta DNA fragments to the adaptors followed by bisulfite conversion was also amplified under the same condition described above. Size distribution of amplified libraries was analyzed using Agilent Bioanalyzer. The results are shown in FIG. 2. (b) For quantification of the post-bisulfite DNA library, the real time PCR reaction was set up as follows: diluted DNA samples from Step a (10,000 fold dilution with water) 1 μl 2X SYBR green PCR master mix (Epigentek) 10 μl, forward PCR primers 1 μl, reverse PCR primers 14 Total reaction volume was adjusted to 20 μl by adding an appropriate volume of water. The following PCR protocol was used: 7 min at 95° C., 30 cycles of 10 sec at 95° C., 10 sec at 55° C. and 12 sec at 72° C. As shown in FIG. 3, PCR products for post-bisulfite DNA library are much higher than that of a DNA library prepared by DNA ligation followed by bisulfite conversion.

What we claim is:

1. A method for preparing a DNA template used for nucleic acid amplification and sequencing reactions to analyze 5-methylcytosine (5-mC) specific DNA methylation status in the template comprising the steps of (a) treating a DNA sample with bisulfite salts; (b) generating a bisulfite-modified, double stranded DNA (dsDNA) from bisulfite-treated DNA sample with a single or a group of appropriate enzymes at an appropriate amount for an appropriate incubation time period in the presence of a group of random primers; (c) ligating said bisulfite modified dsDNA to an adaptor to form adaptor-bisulfite modified dsDNA constructs; and (d) amplifying said adaptor-bisulfite modified dsDNA constructs.

2. The method according to claim 1, wherein said DNA sample is derived from a cell, tissue, organism, or body fluid.

3. The method according to claim 1, wherein said treating a DNA sample with bisulfite salts is accomplished by processing the DNA sample with sodium bisulfite.

4. The method according to claim 1, wherein said appropriate enzymes are selected from phi29 DNA polymerase, Bst DNA polymerase, exonuclease deficient Klenow DNA polymerase, T4 DNA polymerase, native and modified T7 DNA polymerase, HIV-1 reverse transcriptase, M-MLV reverse transcriptase and AMV reverse transcriptase.

5. The method according to claim 1, wherein said random primers are selected from a group of oligos with length of 4 mers to 20 mers.

6. The method according to claim 1, wherein said an appropriate incubation time period is from 5 min to 10 hours.

7. A method according to claim 1, wherein said adaptor is an oligonucleotide having 20-80 bps in length.

8. The method according to claim 1, wherein said adaptor-bisulfite DNA constructs have 200-500 bps in length.

9. The method according to claim 1, wherein said adaptor-bisulfite DNA constructs are subjected to amplification using adaptor-binding specific primers.

10. The method according to claim 1, wherein said amplification is carried out by use of at least one selected from a group consisting of an isothermal amplification method, an LCR method, an Methylation Specific PCR (MSP) method, a nested MSP method, a HeavyMethyl™, real time PCR method, MSP MethyLight™ method, MethyLight™ method, methylation-specific high resolution melting (MS-HRM) method, Headloop MethyLight™ method, MSP Scorpion™ method, and Methylation-sensitive Single Nucleotide Primer Extension (Ms-SNuPE) method, and combinations thereof.

11. The method according to claim 1, wherein said sequencing reaction is carried out by use of at least one selected from a group consisting of 454 pyrosequencing, Illumina (Solexa) sequencing, SOLiD sequencing, and Ion semiconductor sequencing.

12. A method for converting bisulfite treated single stranded DNA (ssDNA) into dsDNA comprising subjecting the said ssDNA to a complement reaction in the presence of DNA-dependent DNA polymerase or RNA-dependent DNA polymerase, a dNTP and random primers.

13. The method according to claim 13, wherein said DNA-dependent DNA polymerase is exonuclease deficient Klenow DNA polymerase.

14. The method according to claim 13, wherein the RNA-dependent DNA polymerase is M-MLV reverse transcriptase.

15. The method according to claim 13, wherein the random primers are random hexamers or random octamers.
